(19) United States
(12) Patent Application Publication
(10) Pub. No.: US 2021/0353459 A1
(43) Pub. Date: Nov. 18, 2021

(54) OXYGEN ENRICHMENT DURING CORNEAL COLLAGEN CROSSLINKING

(71) Applicant: OREGON HEALTH & SCIENCE UNIVERSITY, Portland, OR (US)

(72) Inventors: David Huang, Portland, OR (US); Omkar Thaware, Portland, OR (US)

(21) Appl. No.: 17/320,028

(22) Filed: May 13, 2021

Related U.S. Application Data
(60) Provisional application No. 63/025,036, filed on May 14, 2020.

(51) Int. Cl. A61F 9/00; A61F 9/007; A61F 9/06; A61M 35/00

(52) U.S. Cl. CPC A61F 9/0026; A61F 9/0079; A61M 2202/0208; A61M 35/00; A61M 2210/0612; A61F 9/068

(57) ABSTRACT

Embodiments of the present disclosure relate to methods and apparatus relating to corneal collagen crosslinking (CXL). In some embodiments, disclosed methods and apparatus provide boosted oxygen diffusion into corneal stroma and make possible efficacious accelerated CXL using a non-contact apparatus that reduces use of disposable supplies. In some embodiments, increased atmospheric oxygen concentration around a corneal outer surface occurs, which increases oxygen diffusion into stroma. Accordingly, in some embodiments, increased CXL efficacy occurs as well as reduced overall procedure time.

### RELATED APPLICATION

This application claims priority benefit of U.S. Provisional Patent Application No. 63/025,036, filed May 14, 2020, which is hereby incorporated by reference in its entirety.

### TECHNICAL FIELD

This disclosure relates generally to corneal collagen crosslinking (CXL), and more particularly, to increasing oxygen diffusion into the corneal stroma by enriching environmental oxygen concentration during CXL to provide improved treatment of corneal ectatic diseases.

### BACKGROUND INFORMATION

Corneal ectatic diseases such as keratoconus, pellucid marginal degeneration, and post refractive surgery ectasia are characterized by progressive distortion in the corneal shape that leads to decreased quality of vision. The progression can be halted by CXL using ultraviolet (UV) irradiation. In CXL, riboflavin can be used as a photosensitizer, which in the presence of oxygen facilitates the creation of reactive oxygen species with exposure to UV light. The reactive oxygen species (specially singlet of oxygen) in turn create covalent bonds between and within corneal collagen fibers, which increases stiffness and stability of the corneal stroma. The increased stiffness and stability can halt the progressive change of corneal shape common to the ectatic diseases discussed above.

In standard CXL procedures (Dresden protocol), the corneal epithelium is removed (e.g., an epi-off step) to provide access to the corneal stroma. A 0.1% riboflavin and 20% Dextran mixed solution is applied topically for 30 minutes to soak through the corneal stroma, followed by, UV-A (ultraviolet A) irradiation at 365-nm wavelength and at 3 mW/cm² irradiance for 30 minutes.

Under the standard CXL procedures (Dresden protocol), it can be difficult for patients to maintain stable gaze at the fixation target for 30 minutes. The extended exposure time also can cause drying of the cornea and ocular surface, which cause patient discomfort and increases postoperative inflammation. Thus, it is desirable to shorten the UV exposure time.

To shorten the exposure time, proportionally higher irradiance levels have been used. Such conventional accelerated CXL procedures, however, produce less corneal stiffening because effective CXL depends on both sufficient oxygen and UV dosage delivered to the corneal stroma. The higher UV irradiance consume stromal oxygen in the process of photopolymerization faster than that in the Dresden protocol, and the reduced procedure time does not allow adequate amount of oxygen to diffuse into the corneal stroma. Thus, increasing the UV irradiance alone is insufficient for effective accelerated CXL because of rapidly consumed oxygen and oxygen delivery becomes the limiting factor.

Previous attempts to increase oxygen diffusion into the cornea during CXL have involved disposables and additional set up times. For instance, Patent Application Publication No. US 2017/0156926 A1 of Friedman et al. describes a mask device worn over both eyes to deliver oxygen gas. The oxygen gas, riboflavin, and UV-A light enters the device through separate openings. The publication asserts that the device increases oxygen concentration above the ambient oxygen concentration.

U.S. Pat. No. 10,010,449 of Lopath describes oxygen enrichment over cornea through a liquid such as perfluorocarbon. An apparatus of a shape and size similar to that of a scleral contact lens is described as maintaining a liquid reservoir over the cornea that contains dissolved oxygen and riboflavin. In order to maintain the higher oxygen concentration over the cornea, liquid of oxygen and riboflavin mixture gets periodically replaced during the UV irradiation. The invention includes an optical element to deliver UV light over cornea, while the apparatus is placed on the eye. These previous inventions involve contact method with cumbersome set up and increase the cost of the procedure because they require more disposable supplies.

### SUMMARY OF THE DISCLOSURE

This disclosure describes techniques to increase environmental oxygen concentration using a non-contact device to boost oxygen diffusion and make efficacious accelerated CXL possible. Aspects of the disclosed techniques entail increasing the atmospheric oxygen concentration around the corneal outer surface, which increases oxygen diffusion into the stroma. Accordingly, disclosed are techniques that, among other things, increase CXL efficacy and reduce procedure time, and do so using a non-contact apparatus that is easier to setup and reduce use of disposable supplies.

Additional aspects and advantages will be apparent from the following detailed description, which proceeds with reference to the accompanying drawings.

### BRIEF DESCRIPTION OF THE DRAWINGS

FIG. 1 is a hybrid side elevation and partly sectional view of components, including a tube, UV light supply, and oxygen gas supply, of a CXL apparatus delivering UV light and oxygen gas to a corneal surface of an eye.

FIG. 2 is a partly sectional view of the CXL apparatus of FIG. 1 showing further details of wide and narrow segments of the tube.

FIG. 3 is a flow chart showing steps of an example CXL procedure using the CXL apparatus of FIG. 1.

FIG. 4 is a graph of oxygen concentration percentage over the corneal surface versus oxygen exposure duration for different oxygen flow rates at a cornea-tube distance of 8 mm.

FIG. 5 is a graph of oxygen concentration percentage over the corneal surface versus oxygen exposure duration for different oxygen flow rates at a cornea-tube distance of 10 mm.

FIG. 6 is a graph of oxygen concentration percentage over the corneal surface versus oxygen exposure duration for different oxygen flow rates at a cornea-tube distance of 14 mm.

FIG. 7 is a bar chart of steady state oxygen concentration as a function of oxygen flow rates and distances between cornea and tube of 8 mm, 10 mm, and 14 mm.

### DETAILED DESCRIPTION OF EMBODIMENTS

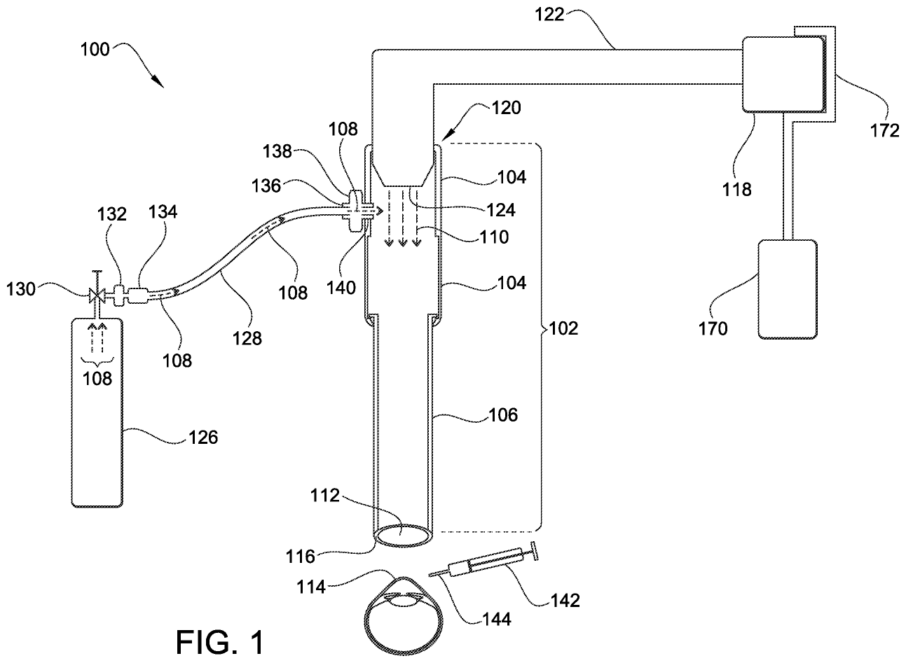

FIG. 1 is a system diagram showing a CXL apparatus 100 to maintain and/or increase stromal oxygen concentration during accelerated CXL by increasing the oxygen concentration at the corneal surface from the normal atmospheric concentration of 21% to a higher level. CXL apparatus 100 includes a hollow tube 102, where hollow tube 102 includes a wide tube segment 104 and a narrow tube segment 106 having different inner widths. For example, in some embodiments, wide tube segment 104 and a narrow tube segment 106 are circular tubing that have respective inner widths (e.g., respective diameters). In other embodiments, wide tube segment 104 and a narrow tube segment 106 are conical or elliptical or rectangular tubing having respective inner widths. In some embodiments, tube 102 is cylindrical or one or both of segments 104 and 106 are cylindrical segments.

Tube 102 delivers oxygen gas 108 and UV light 110 via an opening 112 to a corneal surface 114 facing opening 112. In some embodiments, wide tube segment 104 and narrow tube segment 106 are both transparent. In some embodiments, narrow tube segment 106 is transparent. Indeed, transparency of one or both of wide tube segment 104 and narrow tube segment 106 can help an operator of CXL apparatus 100 view corneal surface 114 through tube 102, which can aid in aligning corneal surface 114 with opening 112 and the delivering of riboflavin solution to corneal surface 114.

In some embodiments, the inner rim diameter of opening 112 is about 14 mm, which is larger than corneal diameter (11-13 mm). In some embodiments, the inner rim diameter of opening 112 is in the range of 11 to 25 mm. This will also reduce the likelihood of contact with cornea. Furthermore, opening 112 includes a smoothed edge or a lower end 116 of narrow tube segment 106. In some embodiments, opening 112 is aligned with corneal surface 114 such that the vertex of corneal surface 114 is centered with opening 112. In some embodiments, opening 112 is located at a distance of approximately 10 to 14 mm above the vertex of corneal surface 114.

In some embodiments, a UV source 118 is attached to an upper end 120 of wide tube segment 104 via a UV delivery system 122. In some embodiments, UV delivery system 122 includes optics such as one or more relay lenses and/or one or more reflecting mirrors that direct UV light from source 118 toward a window 124 and/or opening 112. For example, an end of UV delivery system 122 having window 124 for outputting UV light from UV source 118 is inserted into upper end 120. UV light is output via window 124 and guided through wide segment 104 and narrow tube segment 106 such that it exits opening 112 and contacts corneal surface 114. In some embodiments, UV delivery system 122 has crossing laser beams to align opening 112 at the appropriate distance above the vertex of corneal surface 114. In some embodiments, UV delivery system 122 has a camera providing real time images to help center opening 112 on the vertex of corneal surface 114. In some embodiments, UV light source 118 is attached to a motorized mount or table 170 via an arm 172. The motorized mount is used to adjust the vertical and horizontal position of the entire system to maintain centration and optimal distance between opening 112 and corneal surface 114 during UV exposure. In some embodiments, the UV light produced by UV source 118 is UV-A light having a wavelength of 365 nm. In some embodiments, the UV light produced by UV source 118 and UV delivery system 122 forms a 10 mm diameter spot on corneal surface with homogenous intensity. In some embodiments, the UV light delivered from UV source 118 to upper end 120 is coaxial to opening 112. For example, UV light 110 is projected from its input into hollow tube 102 at upper end 120 along an axis that is coaxial to opening 112 such that UV light 110 exits opening 112 along the axis. In some embodiments, when coaxially aligned in such a manner, UV light 110 exits opening 112 with no or substantially no interreference from an inner surface of hollow tube 102.

In some embodiments, a gas source 126 is attached to wide tube segment 104 via tubing 128 and outputs gas 108 to an aperture 140 of wide tube segment 104. For example, aperture 140 may be a receptacle. In some embodiments, the receptacle provides a connection point of tube 102 to which gas source 126 is coupled. For example, the receptacle allows attachment of tubing 128 to tube 102 such that leakage of gas 108 is reduced. In some embodiments, aperture 140 is located on a side of wide tube segment 104, and therefore, a side of hollow tube 102. In some embodiments, gas source 126 is a liquid oxygen tank that outputs gas 108 in the form of compressed oxygen gas (99% purity). For example, gas 108 in the form of oxygen is output by gas source 126 to wide tube segment 104. The oxygen flows through wide tube segment 104 and narrow tube segment 106 such that it exits opening 112 and contacts corneal surface 114. In some embodiments, a regulator 130 controls the flow rate of gas 108 output by gas source 126. In some embodiments, one or both of a humidifier 132 and a water trap 134 are attached to tubing 128 and humidify gas 108 output by gas source 126. For example, one or both of a humidifier 132 and a water trap 134 humidify oxygen output by gas source 126 such that the oxygen has increased moisture (e.g., 30-32 mg/dL²), which helps reduce drying of corneal surface 114 and corneal thinning during CXL. In some embodiments, tubing 128 is flexible tubing. In some embodiments, tubing 128 is attached to wide tube segment 104 via one or both of a connector 136 and an air filter 138. For example, air filter 138 helps remove microbial contamination of gas 108. In some embodiments, gas source 126 and UV source 118 are both coupled to the same aperture. In some embodiments, tube 102 includes a lumen through which gas 108 is configured to flow toward opening 112. In some embodiments, the lumen defines a longitudinal axis extending along a propagation path of UV light 110 for its incidence upon a corneal surface 114 concurrently with gas 108 to increase gas (e.g., oxygen) concentration at corneal surface 114 to a level exceeding a nominal gas (e.g., oxygen) concentration.

In some embodiments, CXL apparatus 100 includes a syringe 142 that delivers riboflavin solution to corneal surface 114. The riboflavin solution is dropped onto corneal surface 114 through a cannula 144 of syringe 142. In some embodiments, the riboflavin solution contains 0.1% riboflavin solution mixed with isotonic saline and an osmotic agent such as 20% dextran, for example.

In some embodiments, CXL apparatus 100 delivers oxygen gas to corneal surface 114 through tube 102, where tube 102 is positioned coaxial to an ultraviolet beam path output via opening 112. Thus, CXL apparatus 100 can thereby increase the oxygen concentration to an increased value that is, for example, greater than a normal atmospheric concentration of about 21%.

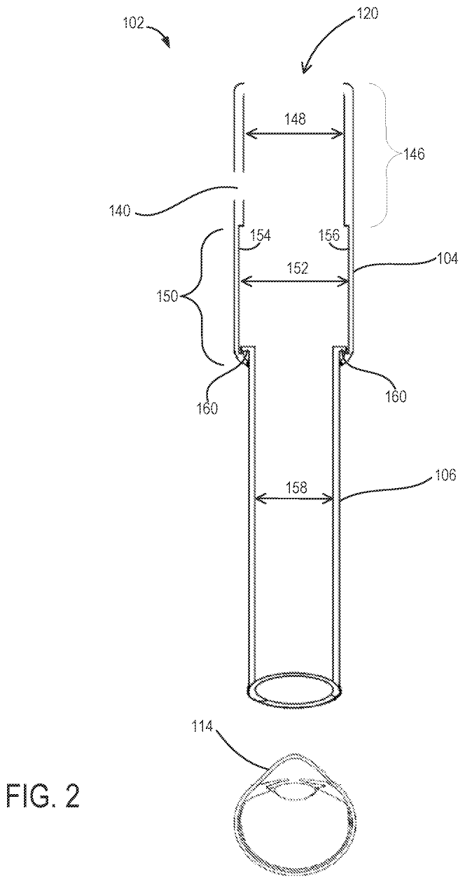

FIG. 2 is a detail view of tube 102 of CXL apparatus 100 of FIG. 1. As discussed above, in some embodiments, tube 102 includes a wide tube segment 104 and a narrow tube segment 106 having respective inner diameters. For example, wide tube segment 104 includes a first section 146 having an inner width 148. For example, inner width 148 is about 14-28 mm. It should be noted that use of “about” in this disclosure encompasses the referenced value exactly and values close to the referenced value. In some embodiments, first section 146 includes aperture 140. In some embodiments, wide tube segment 104 includes a second section 150 having an inner width 152. For example, inner width 152 is about 16-30 mm. Inner width 152 defines the distance between a sliding groove 154 and a sliding groove 156 in second section 150. In some embodiments, grooves 154 and 156 may form a single unified groove that encircles the inner circumference of second section 150. In some embodiments, the upper edge of the narrow tube segment 106 is slidably mounted inside second section 150 of the wider tube segment 104, which may allow for lengthwise adjustment of the hollow tube along a longitudinal axis.

In some embodiments, narrow tube segment 106 has an inner width 158 of about 12 to 26 mm. Narrow tube segment has an inner diameter larger than the UV beam (e.g., larger than a diameter of the UV beam). In some embodiments, narrow tube segment 106 includes a lip 160 that is slidable in sliding grooves 154 and 156 toward and away from upper end 120. In some embodiments, sliding grooves 154 and 156 may define a smooth surface over which lip 160 may travel. For example, narrow tube segment 106 can slide upward via lip 160 in grooves 154 and 156, away from corneal surface 114, in case of accidental contact with the eye or a patient's face. In some embodiments, this sliding ability may reduce likelihood of blunt injury and/or improve safety.

In some embodiments, one or more of the components of CXL apparatus 100 are reusable. For example, tube 102 is usable for multiple patients, and need not be disposed of after each procedure.

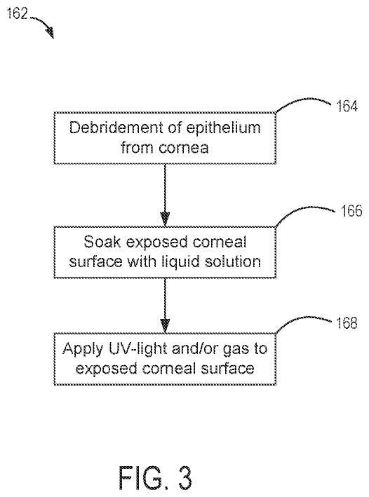

FIG. 3 is a flow chart 162 showing exemplary steps of a CXL procedure using CXL apparatus 100 in accordance with embodiments of the present disclosure. In step 164, removal of epithelium from the outer surface of a cornea to be treated. In some embodiments, the epithelial removal area is 8 to 10 mm in diameter. In some embodiments, epithelial removal is performed with a blunt spatula. However, in other embodiments, an excimer laser or alcohol solution could be used for epithelial removal.

In step 166, the exposed corneal surface is soaked with a riboflavin solution. As discussed above, in some embodiments, the riboflavin solution contains 0.1% riboflavin solution mixed with isotonic saline and an osmotic agent such as 20% dextran, for example. In some embodiments, one drop of the riboflavin solution with 20% dextran is applied to the exposed corneal surface every 2 minutes over a period of 30 minutes. In some embodiments, the riboflavin solution is delivered from syringe 142 through cannula 144 of syringe 142.

In step 168, UV light 110 and oxygen gas 108 is applied to the exposed corneal surface 114. In some embodiments, the operative eye is held open with a speculum and centered under opening 112 of CXL apparatus 100. In some embodiments, corneal surface 114 is positioned about 10 mm to about 16 mm from opening 112 through which both the UV light and oxygen gas is delivered.

Oxygen gas 108 is turned on through, for example, regulator 130 to obtain a desired flow rate in CXL apparatus 100. In some embodiments, the flow rate is 1 liter/minute (L/min). In some embodiments, the flow of gas 108 is maintained for about 2 minutes to about 3 minutes to allow for filling of wide tube segment 104 and narrow tube segment 106, exiting of gas 108 via opening 112, and enriching concentration of oxygen gas 108 over outer corneal surface 50.

UV light 110 is provided by UV light source 118 to irradiate corneal surface 114 for a period of time. In some embodiments, the UV light is UV-A light of 365 nm wavelength. In some embodiments, the irradiance amount is about 3 mW/cm². In some embodiments, the duration of UV irradiation is about 30 minutes. However, in other embodiments, an accelerated UV regimen may be implemented where higher irradiance is applied to corneal surface 114 over a shorter time period; for example, 9 mW/cm² for 10 minutes. In other embodiments, a pulsed UV regimen may be implemented where, for example, corneal surface 114 is exposed to pulsed UV light 110 (e.g. 1 minute UV light on and 1 minute UV light off at 18 mW/cm² for total 10 minutes or 5 cycles).

In some embodiments, a drop of the riboflavin solution discussed above (e.g., 0.1% riboflavin solution mixed with isotonic saline and an osmotic agent such as 20% dextran) is applied for every interval of about 2 minutes to about 5 minutes during UV irradiation.

At the conclusion, UV light 110 and oxygen gas 108 are shut off. The speculum is removed. In some embodiments, a bandage soft contact lens is place on the eye and antibiotic and anti-inflammatory medications are applied topically.

### In Vitro Experiments

An in vitro experiment was performed with a setup includes, the same or substantially the same elements as CXL apparatus 100 of FIG. 1. An oximeter was used to measure oxygen gas concentration over the surface of a contact lens (used to simulate cornea) placed on a face mannequin.

Enrichment of oxygen concentration above the cornea (e.g., simulated by contact lens) was tested using a range of operating parameters. One parameter was the cornea-tubing distance measured between lower end 116 of narrow tube segment 106 and the top of corneal surface 114. The three cornea-tubing distances tested were 8 mm, 10 mm and 14 mm. Another tested parameter was oxygen flow rate. Flow rates of 0.25 L/min, 0.50 L/min and 1 L/min were tested.

Concentration of oxygen gas over the surface of corneal surface 114 was measured in percentage by oximeter having a suction rate set at about 0.1 L/min. Oximeter was a Unitec RM 8000 with a sampling tube having a 1.50 mm inner diameter placed 3 mm above the surface of corneal surface 114.

Oxygen gas was released from oxygen source for 4 min and 30 secs for each experimental trial. The oxygen concentration was measured manually for every 30 second interval, starting from the time oxygen source 126 was turned on until the measured oxygen concentration returned to baseline after gas was turned off. The measured baseline oxygen concentration (e.g., environmental level) was 20.9%.

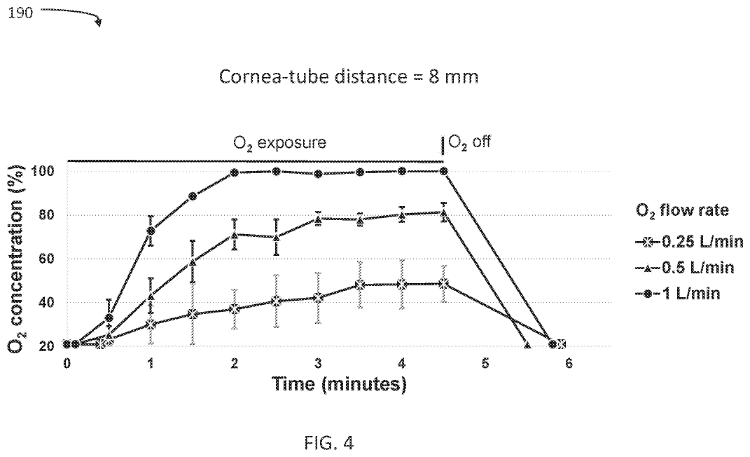

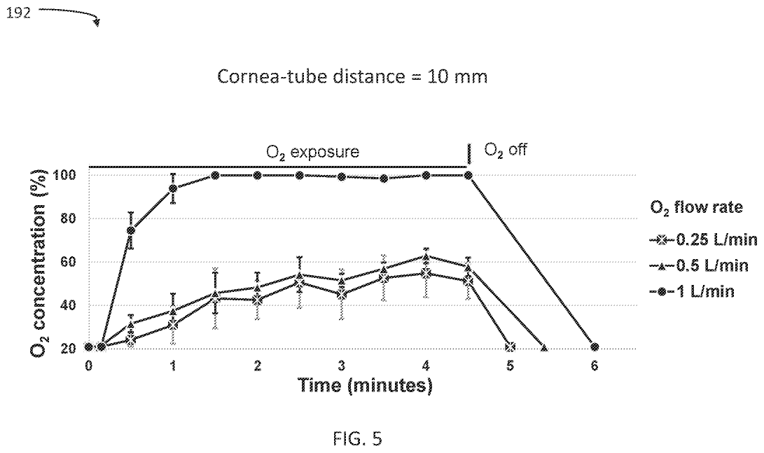

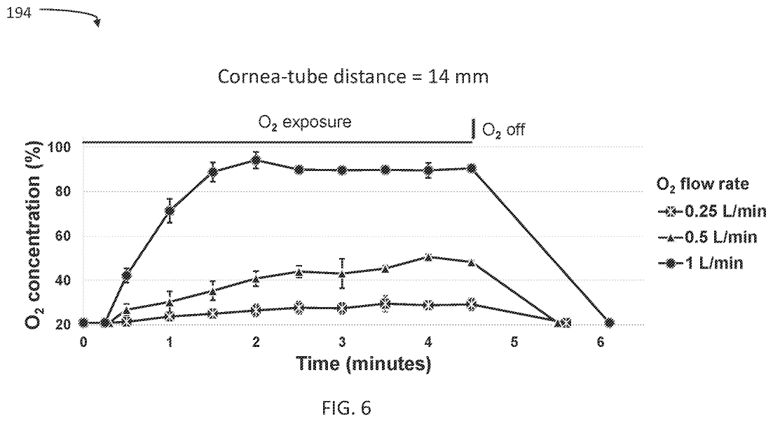

FIGS. 4-6 include graphs 190, 192, and 194 showing oxygen concentration percentage versus oxygen exposure duration for different oxygen flow rates at cornea-tube distances of 8 mm, 10 mm, and 14 mm. Graphs 190, 192, and 194 show the time course of oxygen concentration over experimental trials and are plotted by taking average concentrations from three trials performed at each setting. The error bars represent the standard deviation of the 3 trials. With reference to graphs 190, 192, and 194, it took approximately 2.5 minutes to 3.5 minutes for oxygen concentration to reach a plateau. After cutting off the oxygen exposure at 4 min 30 secs, it took an average of 1 minute to 2 minutes for oxygen concentration to fall back to baseline. The highest oxygen concentration was achieved at a 1 L/min oxygen flow rate in each of graphs 190, 192, and 194.

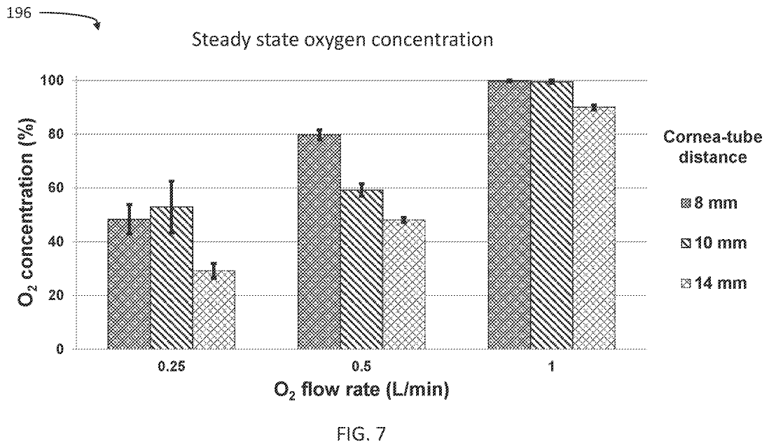

FIG. 7 is a graph 196 showing oxygen concentration percentage versus oxygen flow rate in relation to corneal-tube distances of 8 mm, 10 mm, and 14 mm. Three trials were conducted for each oxygen flow rate (0.25 L/min, 0.50 L/min and 1 L/min) and distance, and the oxygen concentrations during the plateau phase of the experiments between 3.5 minutes to 4.5 minutes (after oxygen flow was turned on) were averaged for each trial. The error bars represent the standard deviation of the 3 trials. As shown by graph 196, there is a trend of decreasing oxygen concentration with increasing cornea-tube distance and lower flow rates. At oxygen flow rate of 1 L/min, greater than 90% oxygen concentration was obtained over the range of 8 mm to 14 mm cornea-tube distances. This range is maintainable under operating room conditions and allows for insertion of a cannula to drop liquid solution onto a corneal surface.

The experiment show that techniques of the present disclosure can consistently enrich oxygen concentration at least three times higher than the environmental oxygen level. This would drive more than 4.3-fold increase in oxygen diffusion rate into the cornea and enable the reduction of UV exposure time by a factor of 3 to 4 and still achieve the same degree of corneal strengthening.

### CONCLUDING REMARKS

Skilled persons will appreciate in light of this disclosure that many changes may be made to the details of the above-described embodiments without departing from the underlying principles of the invention. The scope of the present invention should, therefore, be determined only by the following claims.

1. A non-contact corneal collagen crosslinking (CXL) apparatus for increasing stromal oxygen concentration during accelerated CXL by oxygen gas and ultraviolet (UV) light delivery to a corneal surface, the apparatus comprising:
a light source to generate the UV light; and
a hollow tube coupled to the light source, the hollow tube having an oxygen gas receptacle, an opening, and a lumen, the oxygen gas receptacle configured to receive a supply of the oxygen gas, the opening being located opposite to where the hollow tube is coupled to the light source and configured to confront the corneal surface, and the lumen configured so that the oxygen gas flows toward the opening, the lumen defining a longitudinal axis extending along a propagation path of the UV light for its incidence upon the corneal surface concurrently with the oxygen gas to increase oxygen concentration at the corneal surface to a level exceeding a nominal oxygen concentration.

2. The non-contact CXL apparatus of claim 1, in which the hollow tube includes first and second segments, the second segment slidably mounted inside first segment to provide lengthwise adjustment of the hollow tube along the longitudinal axis.

3. The non-contact CXL apparatus of claim 2, in which an upper end of the second segment comprises a lip that is slidable in the first segment.

4. The non-contact CXL apparatus of claim 2, in which a lower end of the second segment has smoothen edges.

5. The non-contact CXL apparatus of claim 2, in which a lower opening of the second segment has an inner diameter in a range of 11 mm to 25 mm.

6. The non-contact CXL apparatus of claim 1, in which the oxygen gas receptacle is an aperture in a sidewall of the hollow tube.

7. The non-contact CXL apparatus of claim 1, further comprising an air filter configured to remove contamination of the oxygen gas.

8. The non-contact CXL apparatus of claim 1, in which at least a portion of the hollow tube is transparent.

9. The non-contact CXL apparatus of claim 1, further comprising a regulator to control a flow rate of the oxygen gas.

10. The non-contact CXL apparatus of claim 1, further comprising a humidifier and a water trap configured to humidify the oxygen gas.

11. A method of increasing stromal oxygen concentration during non-contact accelerated corneal collagen crosslinking (CXL), the method comprising:
generating a flow of oxygen gas within a lumen of a non-contact CXL apparatus, the non-contact CXL apparatus having an opening for confronting an exposed corneal surface;
generating UV light along a propagation path aligned with the exposed corneal surface; and
simultaneously outputting the flow of the oxygen gas and the UV light through the opening and toward the exposed corneal surface to increase oxygen concentration at the exposed corneal surface to a level exceeding an ambient oxygen concentration.

12. The method of claim 11, further comprising aligning a vertex of the exposed corneal surface with the opening of the non-contact CXL apparatus through which the UV light propagates and the oxygen gas flows from.

13. The method of claim 11, in which a distance between the opening and a corneal vertex is in a range of 8 mm to 14 mm.

14. The method of claim 11, in which a flow rate of the oxygen gas is between 0.5 L/min and 2 L/min.

15. The method of claim 11, further comprising humidifying the oxygen gas before it is output through the opening.

16. The method of claim 11, further comprising filtering the oxygen gas before it is output through the opening.
